(19) United States
(12) Patent Application Publication
(10) Pub. No.: US 2021/0151166 A1
(43) Pub. Date: May 20, 2021

(54) Stress Reduction System and Method

(71) Applicant: Ulrich Schmid, Spartanburg, SC (US)

(72) Inventor: Ulrich Schmid, Spartanburg, SC (US)

(21) Appl. No.: 16/683,562

(22) Filed: Nov. 14, 2019

(51) Int. Cl. G16H 20/70; G16H 20/60

(52) U.S. Cl. CPC G16H 20/70; G06F 3/0482; G16H 20/60

(57) ABSTRACT

A stress reduction system includes a plurality of devices designed to relieve anxiety through the five senses: smell, touch, sight, sound and taste. The system preferably takes the form of a kit, wherein each of the devices works on at least one of the primary senses in a therapeutic and relaxing manner to calm a person and place them more at ease in a stressful environment. A downloadable software “app” on a phone or tablet may include instructions on the proper use of the various relaxation devices in the kit, as well as breathing instruction, meditation techniques, self-hypnosis steps and instruction, or other types of mental or therapeutic exercises that are used to calm the body and the mind. The kit may include a variety of other sensory items, such as ear buds, cooled eye shades, foods with natural extracts used for relaxation, and aromatic pins.

### BACKGROUND OF THE INVENTION

Anxiety, stress and panic attacks are enormous problems across the world today, and treatment for those issues is currently a multi-billion dollar industry. Certain activities tend to cause additional stress in many people, and one major source of anxiety for millions of people is commercial air travel. Between extensive TSA security measures, flight delays, cancellations and the general anxiety associated with airline accidents and terrorism, commercial air travel is one of the most commonly cited causes of stress and anxiety today. Additionally, other types of activities that cause anxiety include being a hospital patient (or a family member or spouse of a hospital patient), dealing with dysfunctional or addicted family members or friends, or even rooting for a hapless college football team that is consistently mired in mediocrity, or worse.

Many types of anxiety treatments are currently available, including both over the counter and prescription pharmaceuticals, alcohol, natural herbal supplements, and various types of systems and methods that are designed to help a person meditate, relax, and reduce levels of stress. The following references describe some of these treatment systems and techniques, and all of these documents are hereby incorporated herein by reference:

U.S. Pat. No. 3,272,198 Method for Inducing State of Reduced Awareness

The present invention is directed to a method of inducing in human beings a state of reduced awareness and loss of pain sensibility, without the use of drugs or electric current.

U.S. Pat. No. 4,315,502 Learning-Relaxation Device

Disclosed is a device for relaxing, stimulating and/or driving brain wave form function in a human subject. The device comprises, in combination, an eye mask having independently controlled left and right eyepieces and a peripheral light array in each eyepiece, an audio headset having independently controlled left and right earpieces and a control panel which controls light and sound signals to the light arrays and earpieces, respectively. Various control functions allow simultaneous or alternating light and sound pulsations in the left and right light arrays and earpieces, as well as selective phasing between light and sound pulsations.

U.S. Pat. No. 4,388,918 Mental Harmonization Process

A state of relaxation or mental harmonization in a subject is created by exposing a color solely to one field of vision of a subject and the complement of that color solely to the other field of vision of the subject while simultaneously exposing an audible tone solely to one ear of the subject and a harmonious tone solely to the other ear of the subject. The color and tones employed are subjectively comfortable and compatible. Preferably, the frequency difference between the two audible tones is one-half the frequency of the audible tone having the lowest frequency.

U.S. Pat. No. 4,553,534 Apparatus for Relieving Psychological Stress

An apparatus for treating stress has a support shaped to fit on the head and constructed to shield the ears from external noise, a display carried on the support and positionable in front of the wearer's eyes, sound transducers in the support positioned to be juxtaposed with the wearer's ears, and a recorder and amplifier for activating the transducers and display with stress-reducing sounds and images. A visor forming the display is pivoted on the support so it can be moved out of the wearer's line of sight. The display itself can be a television-type screen, a multiplicity of light-emitting diodes, or a transparency or slide-type projection arrangement. The support can be a head-encasing helmet of the type worn in football or could be a pair of ear-covering stereophonic headphones carrying a visor provided with the display.

U.S. Pat. No. 4,777,937 Mood Altering Device

The invention features a method and apparatus for altering the mood of an individual. A facial mask is provided that excludes external stimuli while providing visual and auditory stimulation designed to influence the brain wave pattern.

The visual stimulus introduces a continuous, uniform light of a particular color. The light stimulus is designed to produce an effect known as ganzfeld, wherein there occurs a complete loss of visual sensation.

U.S. Pat. No. 6,267,721 Method and Apparatus for Stress Relief System

A stress relief system having a headset to be removably worn on a person's head and a portable electrical wave generator assembly. The left and right speakers of the headset are connected to an electricity powered device for playing recorded music. The electrical wave generator assembly is powered by a 9 volt D.C. battery and it has two closed electrical circuits for transmitting that receive an electrical wave from the electrical wave generator. Each of these circuits has a light emitting diode (LED) that radiates light in the infrared and visible nanometer range. The structure for supporting the respective LED's would be a predetermined length of double stranded wire having sufficient flexibility so that it can be bent to direct the light at preselected points on a patient's hands that are identified by the Koryo Hand Therapy Meridians. A finger loop strap is used to locate and support the tights on the user's fingers.

U.S. Pat. No. 6,702,767 Multisensory Stimulation System and Method

A multisensory stimulation system and method of use. The system includes a housing articulate on a support so as to align a marked viewport with an individual. The housing defines a viewing chamber which is an extension of a reflective light and image optical system chamber which receives light and images from one or more video monitors or images projectors and, in some embodiments, from alternate sources such as black lights and strobe light devices. The system also includes an aromatic sensory component, tactile sensation devices, an audio input system, and audio delivery devices.

U.S. Pat. No. 8,529,457 System and Kit for Stress and Relaxation Management

This invention relates to a system and a kit for stress and relaxation management. A cardiac activity sensor (101) is used for measuring the heart rate variability (HRV) signal of the user and a respiration sensor (102) for measuring the respiratory signal of the user. The system contains a user interaction device (103) having an input unit (104) for receiving user specific data and an output unit for providing information output to the user. A processor (106) is used to assess the stress level of the user by determining a user related stress index. The processor is also used to monitor the user during a relaxation exercise by means of determining a relaxation index based on the measured HRV and respiratory signals, the relaxation index being continuously adapted to the incoming measured signals and based thereon the processor instructs the output unit to provide the user with biofeedback and support messages. Finally, the processor uses the user specific data as an input in generating a first set of rules defining an improvement plan for self-management of stress and relaxation. The first set of rules is adapted to trigger commands instructing the output unit to provide the user with motivation related messages. Also, at least a portion of said user specific data is further used to define a second set of rules indicating the user's personal goals.

U.S. Pat. No. 10,186,163 System and Method for Reducing Stress and/or Pain

A stress reduction and/or pain reduction method and system, includes measuring an initial level of user stress, which level is unacceptable, having the user hold a card having multiple single digit math problems therein, the problems being in multiple series of four problems, the math problems comprising at least one of addition, subtraction, and multiplication problems, displaying to and instructing the user to complete a first series of four and only four problems correctly, displaying to and instructing the user to complete a second series of four and only four problems incorrectly, again measuring the user's stress level and repeating the steps of displaying and instructing, and measuring, until the output from the device is acceptable. The same process may be followed using a camera or video camera and analyzing the image(s) for a Duchenne smile, in place of or in addition to measuring the indicator of stress.

However, each of the references cited above describe system and/or devices that suffer from their own disadvantages, complexities and drawbacks. It would be desirable to provide a small travel kit that incorporates a system and method for relieving stress and anxiety, and which is simple to use, inexpensive to manufacture, and which targets all five senses simultaneously to provide a more relaxing, enjoyable travel experience. It should be understood that while the present stress reduction system and method is described herein by using examples of travel circumstances, the system and method may be used in any other circumstance, and is not intended to be used exclusively in connection with travel. Other desired uses are contemplated and encouraged for any situation in which a user is attempting to achieve a fully relaxed, peaceful and calm state.

### BRIEF SUMMARY OF THE INVENTION

In accordance with one aspect of the invention, a first embodiment of a stress-reduction system and method includes a kit having a variety of devices that may be used simultaneously and therapeutically to reduce anxiety, particularly while traveling, during a hospitalization, or while engaged in any type of stressful activity. Such a system may include a downloadable software application (also referred to herein as an “app”) for a smart phone, tablet, or other computing device that ties the system together, provides instruction for using the other components, and may include relaxing sounds, reading materials, and the like.

Other elements of the system may include eye shades, audio ear buds or headphones that work with the app, ear plugs, aroma pins to provide relaxing smells to stimulate olfactory senses, and mints or candy. Rubber or foam squeeze balls or other squeezable items may be included, along with activity-specific items, such as information cards about travel (for travel kits), cards with inspirational messages or bible quotes (for hospital kits, for instance), or even a standard deck of playing cards to be used as a diversionary activity. There are many types of hand-held vibrating body massagers that may be included, as well as other types of relaxation devices, such as the wildly popular fidget spinners, massage rollers, travel pillows, weighted blankets, body-cooling towels, acupressure devices such as slippers, wristbands or socks, and the like.

In one embodiment, the eye shades may also be incorporated with an instant-cold packet, which may be inserted into the eye shades, or the eye shades may be manufactured with an instant-cold packet therein and designed for single use. Instant cold-packs are commonly available today, and typically include ingredients that, when mixed together, produce an endothermic reaction that causes the packet to get cold quickly. In this embodiment of the eye shade, the instant pack is incorporated into the eye shades, so that a user can initiate the chemical reaction of the packet within the eye shades and then place the eye shades on his or her face over the eyes and/or forehead, which provides not only shade from light, but also provides a cool, relaxing feeling over the eyes and the facial area surrounding the eyes. The cooled eye shades produce what is commonly referred to as a “dive effect,” which tends to calm a person's heart and breathing rates, and has a generally positive influence on a user's mindset and general sense of well-being.

In use, a user simply accesses the app on her phone, tablet or computing device, puts on the headphones or earbuds that are operatively connected to and controlled by the computing device, and follows instructions for using the various components of the kit. The instructions may include proper placement or use of the acupressure devices and other items, and once the user has all of the items functioning in the proper manner, the app may continue to provide relaxing and soothing sounds. Other features are described in more detail below.

### BRIEF DESCRIPTION OF THE DRAWINGS

These and other features, aspects, and advantages of the present invention will become better understood with regard to the following description, appended claims, and accompanying drawings where:

FIG. 1 is a perspective view of one embodiment of a stress reduction system in the form of a kit, wherein the kit includes devices to provide stress relief through each of the five senses: a mobile smart phone and ear buds for visual and audio stimulation, a vial containing a scented oil or extract, a fidget spinner and hand-held massager, a pair of eye shades and a cool pack for insertion into the eye shades, and a bag of edible items;

FIG. 2 is a perspective view of one embodiment of a stress reduction system in use by a user, wherein the user is wearing a pair of cooled eye shades and ear buds connected to a phone, and the user is applying a hand-held massager against her arm, and further showing a fidget spinner, a vial of scented oil or extract, and an edible item having stress relieving properties.

### DETAILED DESCRIPTION OF THE INVENTION

The present invention includes, in a first embodiment, a stress reduction system that includes a variety of devices designed to relieve anxiety through the five senses: smell, touch, sight, sound and taste. The system preferably takes the form of a kit, wherein each of the devices works on at least one of the primary senses in a therapeutic and relaxing manner to calm a person and place them more at ease in a stressful environment, such as a hospital, an airplane, or the like.

In one embodiment, the system is tied together by an app on a phone 10, tablet or other computing device. The app provides instructions for the user to follow, either visually or audibly through ear buds 12 or headphones. Specifically, the app may include instructions on the proper use of the various relaxation devices in the kit. Additionally, the app may include a menu of other types of calming exercises for the user to select. For example, some of the menu choices may include breathing instruction, meditation techniques, self-hypnosis steps and instruction, or other types of mental or therapeutic exercises that are used to calm the body and the mind.

The kit preferably includes headphones or ear buds 12 that may be operatively connected to the phone 10 or tablet (either with a cord, or via wireless connection such as Bluetooth or wifi), and the ear buds 12 are used for the auditory portion of the relaxation program. As stated previously, the ear buds 12 may be used to receive various types of instructions, and the app may also include a menu of soothing sounds or music. For instance, a sound menu on the app may include soothing sounds such as a babbling brook, a crackling fire, waves lapping up onto a beach, forest noises (breezes through the leaves, birds and crickets chirping, etc.) or other similar types of relaxing sounds. Additionally, the app may include relaxing music choices, as well, including anything from classical music to chants by the Gregorian Monks. Other types of audio stimuli may include short stories or novels (audio books), and the app may further include the capability for the user to download specific sounds or other specifically desired auditory stimuli.

The app may also include relaxing visual stimuli, such as slide shows or videos of quiet, peaceful environments, or other types of calming images. In some embodiments, the visual stimuli may be synchronized in any suitable manner to the music or other audio stimuli provided by the app. In another embodiment, the stress reduction kit may include a set of virtual reality goggles for viewing the visual stimuli as 3-dimensional images.

The stress reduction kit may also include eye shades 14 for a user to wear for blocking light. In one embodiment, the eye shades 14 may incorporate therein an instant-cold packet 16, which turns the eye shades 14 cold upon activation. The instant-cold packet 16 may be either inserted into an internal portion of the eye shades 14 (similarly to a pillow sliding into a pillowcase), or the instant-cold packet 16 may be permanently incorporated therein and used as a single-use device. The eye shades 14 provide not only shade from ambient or direct light, but also provide a cool, relaxing feeling over the eyes and the facial area surrounding the eyes. Alternatively, a re-usable cold pack may be used in association with the eye shades, where a user places the re-usable cold pack (or a pair of eye shades having a re-usable cold pack positioned therein) into a freezer, ice box, or refrigerator prior to use.

For olfactory relaxation, aromatic pins 18, vials of scented substances, or other types of aromatic devices may be used. It is well known that certain scents are used for relaxation and calming purposes. For instance, peppermint is commonly used as an energy-booster, lavender is used to induce relaxation, and jasmine is traditionally used as a sleep aid. Any desired scent may be used, either in the form of a small vial, a pin, perfume bottle, dropper or in any other device that allows a user to smell the scent.

The stress reduction kit preferably includes edible stimuli 20 for the sense of taste, which may include candy such as mints, chocolate or dark chocolate, or may include something that not only has a pleasant and relaxing taste, but also includes calming ingredients, such as melatonin, lavender or cannobidiol (CBD). Other types of tasty relaxation agents may include packets of tea, such as green tea, chamomile, or the like, which are widely used for their calming effects. Other types of candies or foods that include or are infused with certain natural relaxing agents or extracts may be included. Such extracts may include lemon balm, passion fruit, valerian, hops, L-theanine, and the like. Any known relaxing agents or extracts may be used for such purposes, and food products containing such extracts are widely available. In some kits, mini-bottles or other small containers of wine or liquor may be included, as well.

For the sense of touch, along with the eye shades 14 as described above, other devices and articles may be included with the stress-reduction kit, such as hand-held massagers 22 and vibrating devices, weighted blankets, tight fitting compression t-shirts or other tight-fitting compression undergarments, fidget spinners 24, squeezable rubber or foam devices, pillows, and the like. Collectively, the devices listed above to address the sense of touch in a relaxing manner may be referred to as “touch relaxation devices.”

In one embodiment, which may be particularly useful for travel, the app may include a function wherein the user enters the amount of time that will be spent on a particular flight, for instance. Once the user enters that information into the app, then the app essentially designs a full cycle of timed relaxation events. For instance, the following example illustrates one such cycle: if a user enters information into the app that his flight will be 5 hours long, then the app may begin with instructions for placement of the various devices (ear buds 12, cooled eye shades 14, acupressure devices, weighted blanket, etc.). Then, the app may tell the user to take a particular dosage of the enclosed edible stimuli 20 (2 CBD gummies, for instance), and then go through some breathing techniques. After a period of time, the app may instruct the user in some meditation techniques, followed by a quiet period of soothing sounds or music. Then, approximately midway through the flight, the app may instruct the user to take another dosage of edible relaxation food 20, such as having a cup of chamomile tea. Toward the end of the flight, the app may instruct the user to stand up and stretch, and then to use the vibrating massager 22 on the user's shoulders and neck area, followed by more upbeat music and instructions to eat an edible stimuli food item 20 having peppermint extract to bring the user back to full alertness in time to disembark from the plane. In this way, the app is able to provide a full cycle of different calming techniques throughout the flight, so that they are as relaxed as possible during most of the flight, and are then brought back to a higher level of alertness and energy when the flight is over. Collectively, such ingestible food items that are used for stress-relief and relaxation may be referred to herein as “calming foods” or “edible stimuli.”

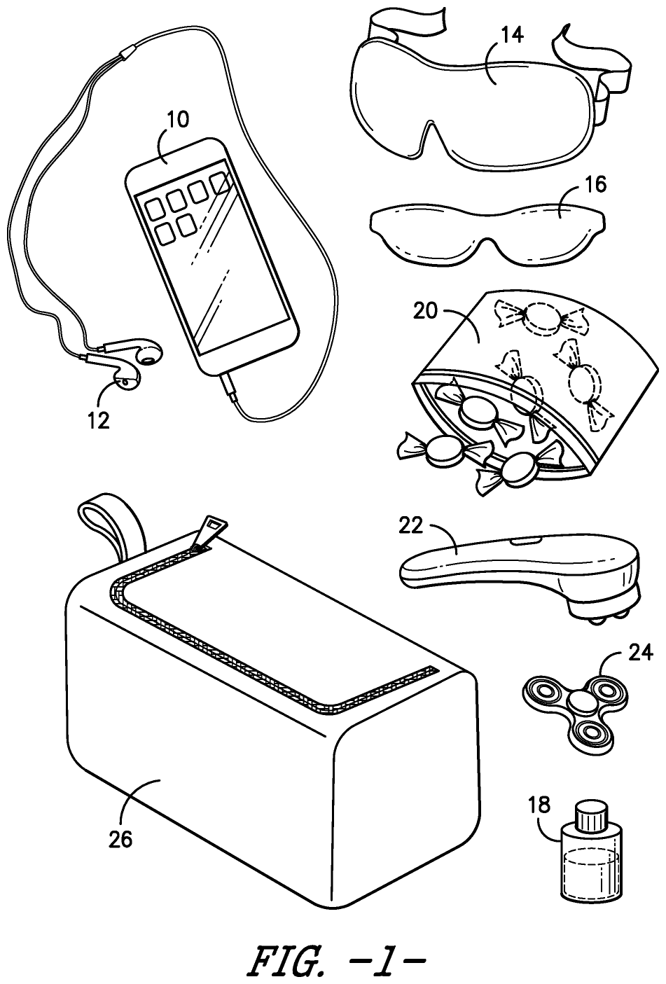

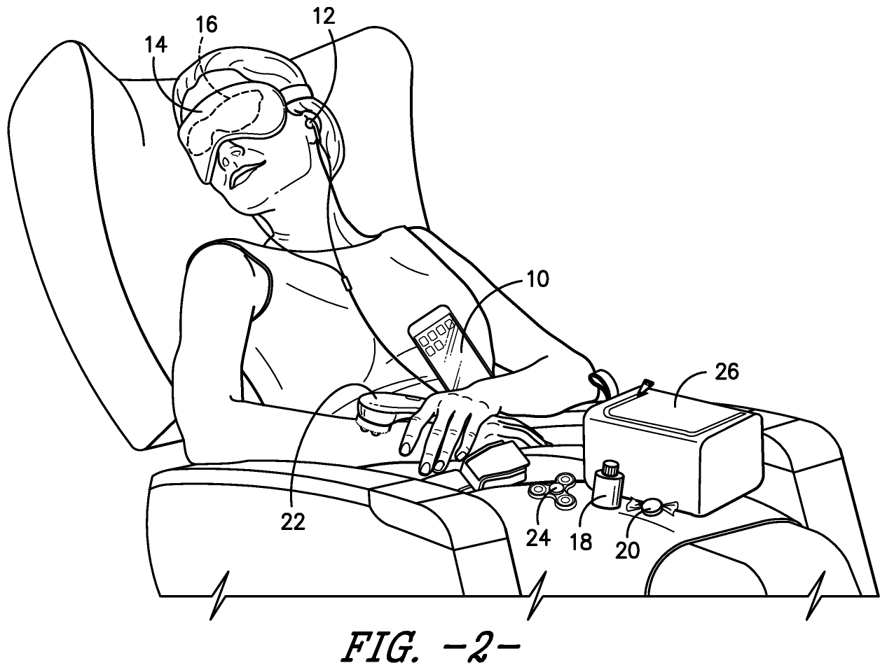

It should be understood that the cycle described above is but one example, and the app may be programmed to do any number of different cycles, based on the preferences of the user and the time of the flight or trip. It should also be understood that the user may instead simply choose different app functions in an a la carte manner. The app menu may allow a user to choose from light relaxation to heavy relaxation cycle, depending on the anxiety level being felt that day by the user. In a preferred embodiment, the stress reduction system may include a carrying case or pouch 26, as shown in FIGS. 1 and 2.

Although the present invention has been described in considerable detail with reference to certain preferred versions thereof, other versions are possible. Therefore, the spirit and scope of the appended claims should not be limited to the description of the preferred versions contained herein. All features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

What is claimed is:

1. A stress reduction system for addressing each of the five senses, including sight, smell, touch, sound and taste, said stress reduction system comprising:
a downloadable software application for a mobile computing device for providing instructions and audio stimuli to a user;
a pair of cooling eyeshades;
an aromatic device for providing olfactory stimuli:
a touch relaxation device; and
a calming food;
wherein said software application provides instructions to a user for using said cooling eyeshades, said aromatic device, said touch stimulation device, and for ingesting said calming food.

2. The stress reduction system set forth in claim 1, wherein said aromatic device is an aromatic pin that includes ingredients selected from the group consisting of peppermint, lavender, and jasmine.

3. The stress reduction system set forth in claim 1, wherein said touch relaxation device is selected from the group consisting of a weighted blanket, a compression undergarment, fidget spinner, squeezable device, a vibrating massager and a pillow.

4. The stress reduction system set forth in claim 1, wherein said calming food is an ingestible food item having extracts selected from the group consisting of lemon balm, passion fruit, valerian, hops, L-theanine, melatonin, lavender and cannobidiol.

5. The stress reduction system set forth in claim 1, wherein said calming food is selected from the group consisting of tea, green tea, chamomile tea, candy, mints, chocolate, and dark chocolate.

6. The stress reduction system set forth in claim 1, wherein said software application includes a menu of audio sounds and stimuli, so that a user may select a preferred audio stimuli.

7. The stress reduction system set forth in claim 1, further including a pair of ear buds that are operatively controlled by said software application for providing said audio stimuli to a user.

8. The stress reduction system set forth in claim 1, wherein said software application is programmed to provide instruction for techniques selected from the group consisting of breathing techniques, meditation, hypnosis, and relaxation methods.

9. The stress reduction system set forth in claim 1, wherein said software application is programmed to provide timed instruction for ingesting multiple doses of said calming foods based on user input of specific travel time.

10. The stress reduction system set forth in claim 1, wherein said software application is programmed to provide instruction for [symbol]
